(19) United States
(12) Patent Application Publication
(10) Pub. No.: US 2009/0035226 A1
(43) Pub. Date: Feb. 5, 2009

(54) USE OF CRANBERRY EXTRACT ENRICHED IN TOTAL PHENOLS AND FREE, ESSENTIALLY FREE, OR SUBSTANTIALLY FREE OF SUGARS, ACIDS, SULFUR AND OTHER CONTAMINANTS, FOR PERIODONTAL TREATMENT

(75) Inventors: Michael S. TEMPESTA, El Granada, CA (US); F. Joseph Daugherty, Omaha, NE (US)

Correspondence Address: HOGAN & HARTSON LLP, ONE TABOR CENTER, SUITE 1500, 1200 SEVENTEENTH ST, DENVER, CO, 80202, US

(73) Assignee: PHENOLICS, LLC, EL GRANADA, CA (US)

(21) Appl. No.: 12/183,943

(22) Filed: Jul. 31, 2008

Related U.S. Application Data
(60) Provisional application No. 60/953,143, filed on Jul. 31, 2007.

(51) Int. Cl. A61K 9/68; A61K 8/34; A61Q 11/00

(52) U.S. Cl. 424/48; 424/49

(57) ABSTRACT

Cranberry and other plant extracts enriched in total phenols and free or substantially free of sugars, acids, sulfur and other contaminants are used for periodontal treatments. The periodontal treatments of the present invention include swish and swallow techniques, in which the extracts enriched in total phenols but free or substantially free of sugars, acids, sulfur and other contaminants, are solubilized in water or other solvent, placed in the mouth, swished around the teeth and then swallowed. Other application techniques include gel applications, gum formulations and toothpaste formulations.

### RELATED APPLICATION

The present application claims priority of U.S. Provisional Application No. 60/953,143 filed Jul. 31, 2007, which is incorporated herein in its entirety by this reference.

### FIELD OF THE INVENTION

The present invention relates to use of plant extracts enriched in phenols for periodontal treatment. More particularly, the present invention relates to the use of extracts from fruits and berries enriched in total phenols to prevent adhesion of oral pathogens from the gums and dentin.

### BACKGROUND OF THE INVENTION

Maintaining oral health depends in large part in controlling the affects of bacteria normally present in the mouth. The bacteria present in the mouth are constantly converting carbohydrates—in particular, sugars and starches—into acids. The mixture of bacteria, acid, food debris, and saliva present in the mouth form plaque, a sticky substance which adheres to teeth. Streptococcus mutans bacteria is a significant contributor to plaque formation. Plaque starts to accumulate on teeth within 20 minutes after eating, and if not periodically removed, tooth decay, gingivitis and peridonitis sets in.

The acids in plaque dissolve the enamel surface of the teeth and create cavities in the teeth. Untreated tooth decay can lead to destruction of the tooth pulp and can ultimately lead to tooth loss.

Periodontal disease, including gingivitis and periodontitis, is a chronic bacterial infection that affects the gums and bone supporting the teeth. Periodontal disease can affect one tooth or many teeth, and can lead to bone loosening and loss. Periodontal disease typically begins when the bacteria in plaque causes the gums to become inflamed. In its initial stages of gingivitis, the gums become red and swollen and bleed easily. Untreated gingivitis may lead to periodontitis, in which plaque spreads to below the gum line. Toxins produced by the bacteria in plaque thereafter stimulate a chronic inflammatory response, and the tissues and bone that support the teeth are broken down and destroyed. Pockets between the teeth and gums become affected, and as the disease progresses, the pockets deepen, and eventually, teeth can become loose and may have to be removed.

Fruits and fruit juices typically contain water, sugars, acids, phenols (including polyphenols), other carbohydrates and salts. By way of example, it is estimated that unsweetened cranberries comprise the followings classes of constituents:

| | |
|---|---|
| 86-90% | water |
| 3-4% | sugars and other simple carbohydrates |
| 3-4% | organic acids (e.g., quinic, malic, citric and benzoic acid) |
| 1-2% | fiber |
| 0.5-1% | total phenols (anthocyanins, proanthocyanins) |

Sweetened cranberry juices contains substantially more sugar.

It has been suggested that cranberry juice may help prevent certain diseases of the gums and teeth by preventing Streptococcus mutans from sticking to teeth. It is postulated that if the bacteria are unable to stick to the teeth, then microbial digestion of sugars and excretion of acid by the bacteria is believed to be lessened, with a concomitant lessening in dental decay also expected. However, it has also been noted that “[t]he sugar that is usually added to cranberry juice can cause cavities, and the natural acidity of the substance may contribute directly to tooth decay.” University of Rochester Medical Center News Archives, Give Thanks for the Cranberry, Say Dental Researchers, Nov. 23, 2005.

The anti-adhesion effects of a cranberry extract on oral bacteria in a series of studies have been postulated to be a result of anti-coaggregating properties. “Dental plaque stability depends on bacterial adhesion to acquired pellicle, and on interspecies adhesion (or coaggregation). A high-molecular-weight cranberry constituent at 0.6 to 2.5 milligrams per milliliter reversed the coaggregation of 49 (58 percent) of 84 coaggregating bacterial pairs tested. It acted preferentially on pairs in which one or both members are gram-negative anaerobes frequently involved in periodontal diseases. Thus, the anticoaggregating cranberry constituent has the potential for altering the subgingival microbiota, resulting in conservative control of gingival and periodontal diseases.” E. Weiss, et al, Inhibiting Interspecies Coaggregation of Plaque Bacteria with a Cranberry Juice Constituent, Journ. Am. Dental. Assoc., December 1998; 129(12); 1719-23.

In a subsequent study the inhibitory effect of a high-molecular-weight constituent of cranberry on adhesion of oral bacteria was confirmed by Weiss, et al: “A high-molecular-weight nondialysable material (NDM) isolated from cranberry juice at a concentration of 0.6 to 2.5 mg/ml dissociated coaggregates formed by many intergeneric oral bacteria. A lower concentration of NDM was required to inhibit formation of such coaggregates. NDM acted preferentially on pairs of oral bacteria in which one or both members are Gram-negative anaerobes. A preliminary clinical trial showed that NDM reduces S. mutans counts in saliva. The antiadhesion activity of cranberry juice has a potential for altering the oral microbial flora resulting in improved oral hygiene.” E. Weiss, et al, Inhibitory Effect of a High-Molecular-Weight Constituent of Cranberry on Adhesion of Oral Bacteria, Crit. Rev. Food Sci. Nutr. 2002; 42(3 Suppl).; 285-92.

In yet a further study by Weiss' group the effect of a mouthwash containing cranberry extract was tested in vivo. “In the present study we determined the effect of mouthwash supplemented with NDM on oral hygiene. Following 6 weeks of daily usage of cranberry-containing mouthwash by an experimental group (n=29), we found that salivary S. mutans streptococci count as well as the total bacterial count were reduced significantly (ANOVA, P<0.01) compared with those of the control (n=30) using placebo mouthwash. No change in the plaque and gingival indices was observed. In vitro, the cranberry constituent inhibited the adhesion of Streptococcus sobrinus to saliva-coated hydroxyapatite. The data suggest that the ability to reduce mutans streptococci counts in vivo is due to the anti-adhesion activity of the cranberry constituent.” E. Weiss, et al, A High Molecular Mass Cranberry Constituent Reduces Mutans Streptococci Level In Saliva And Inhibits In Vitro Adhesion To Hydroxyapatite, FEMS Microbiol Lett. Mar. 12, 2004; 232(1):89-92.

A study was also conducted to reveal mechanisms of antimicrobial activity of selected metabolites of berry-derived phenolic compounds and organic acids. After testing phenolic acids and other acids, it was confirmed that numerous phenolic acids and other acids derived from berries present are microbial metabolites. H-L Alakomi, et al, Weakening of Salmonella with Selected Microbial Metabolites of Berry-Derived Phenolic Compounds and Organic Acids, Journ. of Agri. and Food Chem., publ. Apr. 17, 2007. The metabolitic activity of acids in wine has not only been confirmed, it has been found that the polyphenols in wine are not responsible for the inhibition of growth of S. mutans. M. Daglia, et al., Antibacterial Activity of Red and White Wine Against Oral Streptocci, Journ. of Agri. and Food Chem., publ. Jun. 5, 2007.

### SUMMARY OF THE INVENTION

The present invention relates to the use of cranberry extracts enriched in total phenols and free, essentially free, or substantially free of sugars, acids, sulfur and other contaminants, for periodontal treatment. The cranberry extracts of the present invention contain low molecular weight, mid molecular weight and high molecular weight phenols obtained from cranberries. The periodontal treatments of the present invention include swish and swallow techniques, in which the cranberry extract, preferably in a solvent or carrier, is placed in the mouth, swished around the teeth and then swallowed. Another preferred technique include swishing and spitting of the cranberry extract/solvent formulation. Yet another preferred technique includes painting of a gel containing the preferred cranberry extracts on the teeth, allowing the gel to remain in place for an extended period of time, and then washing the gel off with a water spray.

In other aspects of the present invention, other fruits and berries naturally high in phenols, are extracted to produce extracts enriched in total phenols and free, essentially free or substantially free of sugars, acids, sulfur, and other contaminants, for periodontal treatments.

### DETAILED DESCRIPTION OF PREFERRED EMBODIMENTS

The techniques of the present invention relate to use of plant material extracts enriched in total phenols and free, essentially free, or substantially free of sugars, acids and non-phenolic compounds present in the original plant material, to reduce and prevent adhesion of oral pathogens from the gums and dentin.

As used herein, the term “extract” refers to any substance derived from a plant source that naturally contains phenolic compounds, including extracts prepared from the whole plant or from various parts of the plant, such as the as seeds, fruits, skins, vegetables, nuts, tree barks, roots, and other plant materials that contain phenolic compounds. Most colored fruits, berries, and vegetables are known to contain phenolic compounds. Examples of plants, fruits, berries, and vegetables that contain phenolic compounds include, but are not limited to, blueberries, bilberries, elderberries, plums, blackberries, strawberries, red currants, black currants, cranberries, cherries, raspberries, grapes, currants, hibiscus flowers, bell peppers, beans, peas, red cabbage, purple corn, and violet sweet potatoes. Most preferred plants are cranberry, cherry, strawberry, blueberry, plum, blackberry, raspberry, grape, apple, chokecherry, mangosteen, pear and bananas.

The raw plant material may be used either as is (wet) or may be dried prior to extraction. Optionally, the raw plant material may be presorted by separating and removing the components low in anthocyanins and proanthocyanidins prior to extraction. An “extract” as defined herein, includes one or many constituents obtained originally from the raw plant material.

As used herein, the terms “phenols” include monomeric, oligomeric and polymeric compounds having one or more phenol groups, and include, but are not limited to, anthocyanins, proanthocyanidins and flavonoids, but do not include phenols having carboxylic or other acid moieties.

As used herein, extracts “enriched in total phenols” are obtained by extracting and purifying one or more berries, fruits, vegetables, flowers or other plant material containing phenolic compounds to collect the high molecular weight (“HMW”) phenols, mid-molecular weight (“MMW”) phenols, and low molecular weight (“LMW”) phenols, without preferentially removing HMW, MMW or LMW phenols, while removing all, essentially all or substantially all sugars, acids (defined herein to include phenolic acids, amino acids, nucleic acids, fatty acids, other organic acids and inorganic acids), sulfur (in whatever form), and other contaminants (defined herein to include cellulose, pectin, proteins, plant sterols, and triglycerides).

As used herein, the term “free” means 1% or less in a formulation. As used herein, the term “essentially free” means 5% or less in a formulation. As used herein, the term “substantially free” means 10% or less in a formulation.

While there is no one definition of molecular weight ranges which constitute LMW, MMW and HMW phenols, as used herein, the LMW, MMW and HMW are considered to range, on average, from 300-600, 600-2400, and >2400, respectively.

The primary active ingredients in cranberry are a family of HMW polyphenols that have shown the ability to interfere with the attachment, effacement and colonization of bacteria via powerful anti-adhesion properties that, in effect, provide a barrier to reduce inflammation and infection caused by the adhesion of oral pathogens. These compounds coat sites in the gingiva as well as the pathogens themselves generating a strong protective barrier. However, and without knowing the precise mechanism of action of the HMW polyphenols with respect to the anti-adhesion properties, it is believed that the presence of LMW and MMW phenols enhance the activity of the HMW polyphenols, as well as exhibit some anti-adhesion properties separately.

It is further noted that in the currently available commercial cranberry preparations, most contain significant amounts of naturally occurring organic acids. Such acids are considered to be active agents, with the presence of such acids problematic, since they can potentially erode dentin, the main structural component of teeth, with chronic use over long periods of time.

Preferably, a swish and swallow formulation of the present invention is in the form of a water rinse formulation and may be used by the patient in the dentist's chair, as well as taken at home. A preferred formulation is a cranberry extract enriched in total phenols and substantially free of sugars, acids, sulfur and other contaminants. The most preferred formulation is a cranberry extract enriched in total phenols and free of sugars, acids, sulfur, and other contaminants. After holding in the mouth an aqueous solution containing a cranberry extract enriched in total phenols and free, essentially free, or substantially free of sugars, organic acids, sulfur and other contaminants for 10 to 30 seconds it is to be swallowed as the ingredients have significant systemic benefits for the treatment and prevention of gingivitis as well.

In one preferred swish and swallow formulation of the present invention, water is mixed with a cranberry extract enriched in total phenols and free of sugars, organic acids, sulfur and other contaminants in a ratio by weight percent of 90:10. Substitution of the cranberry extract enriched in total phenols and free of sugars, organic acids, sulfur and other contaminants with a cranberry extract enriched in total phenols and essentially free or substantially free of sugars, organic acids, sulfur and other contaminants is also acceptable. Ranges of acceptable ratios by weight percents each of these cranberry extracts are from 99:1 to 90:10. Other constituents may be added to the swish and swallow formulations, provided (a) the weight percent of the cranberry extract is from 1-10% and (b) only de minimus amounts (i.e., less than 0.5 weight percent) of any sugar, organic acid or sulfur are present in the final formulation.

Other preferred formulations containing the preferred and most preferred formulations described above include
Swish and swallow washes combination with other anti-inflammatory compounds such as CoQ10, vitamin C, vitamin E, and anti-microbial agents;
Higher strength chewing gums;
Lozenges;
Prophy pastes; and
Toothpastes prescribed by the dentists (e.g., periopastes).

A gel formulation is a higher potency formulation of either the preferred formulation or the most preferred formulation described above, solubilized or carried in a gel formulation, to be applied chair side in a tray for an extended treatment of 5-30 minutes, and then removed with a water spray, for spitting or dental evacuation. It is recommended that the gel treatment be followed with a take home swish and swallow rinse. In an alternate embodiment, the gel is formulated with an anti-microbial agent in a single use syringe.

In another gel formulation, the gel is less viscous and is injected directly into sub-gingival pockets. The sub-gingival gel may also be combined with an antimicrobial agent.

A preferred gel formulation includes 1-10% by weight of a cranberry extract enriched in total phenols and free, essentially free, or substantially free of sugars, organic acids, sulfur and other contaminants, and 99-90% by weight of a conventional gel. A known formulation which is contemplated contains ingredients from the gel formulation of U.S. Pat. No. 4,925,654, said patent being incorporated herein in its entirety by this reference. In an embodiment of a gel formulation of the present invention, which is preferably 10% by weight of cranberry extract enriched in total phenols and free, essentially free, or substantially free of sugars, organic acids, sulfur and other contaminants, 90% by weight comprises the gel formulation of Example 2 of U.S. Pat. No. 4,925,654, in approximately the proportions described, with preferably, the required ingredients include sorbitol, polyethylene glycol, water, glycerin, TKPP, TSPP, silicon dioxide, synthetic silica, sodium lauryl sulfate, sodium hydroxide, IOTA Carrageenan gum, Gantrz S-97, sodium benzoate, titanium dioxide and sodium fluoride, or equivalents.

A preferred toothpaste formulation includes 1-10% by weight of a cranberry extract enriched in total phenols and free, essentially free, or substantially free of sugars, organic acids, sulfur and other contaminants, and 99-90% by weight of a conventional toothpaste. A known formulation which is contemplated contains ingredients from the toothpaste formulation of U.S. Pat. No. 4,925,654, except that an alternative sweetener to sodium saccharin (e.g., sucralose) is contemplated. In a contemplated embodiment of a toothpaste formulation of the present invention, which is preferably 10% by weight of cranberry extract enriched in total phenols and free, essentially free, or substantially free of sugars, organic acids, sulfur and other contaminants, 90% by weight comprises the toothpaste of Example 3 of U.S. Pat. No. 4,925,654 (utilizing sucralose instead of sodium saccharin), with most preferably, the required ingredients being water, glycerin, silicon dioxide, TKPP, TSPP, Gantrez S-97, synthetic silica, sodium lauryl sulfate, sodium hydroxide, xanthan gum, sodium benzoate, titanium dioxide and sodium fluoride, or equivalents.

A preferred lozenge formulation includes 1-10% by weight of a cranberry extract enriched in total phenols and free, essentially free, or substantially free of sugars, organic acids, sulfur and other contaminants, and 99-90% by weight of a conventional sugar-free lozenge. A known formulation which is contemplated contains ingredients from the Example 5 lozenge formulation of U.S. Pat. No. 4,925,654, with sucralose or other non-sugar sweetening substituted for the sodium saccharin. In a contemplated embodiment of a lozenge formulation of the present invention, which is preferably 10% by weight of cranberry extract enriched in total phenols and free, essentially free, or substantially free of sugars, organic acids, sulfur and other contaminants, 90% by weight comprises sucralose, magnesium stearate, PEG (4) sorbitan diisostearate, sodium fluoride, Gantrez S-97, TKPP, and TSPP, the previous ingredients preferably collectively comprising 8-9 wt. % of the 90% and sorbitol constituting the remaining wt % to 90% wt % of the preferred formation.

A preferred chewing gum formulation includes 1-10 parts of a cranberry extract enriched in total phenols and free, essentially free, or substantially free of sugars, organic acids, sulfur and other contaminants, 10-50 parts gum base, 3-10 parts binder, 80 parts sorbitol, 0.1 to 5 parts sucralose, 3.5 to 8 parts TKPP:TSPP 3:1, 0.1 to 1 part Gantrez S-97, and 0.1 to 0.05 parts sodium fluoride.

A preferred prophy paste formulation comprises 1-10% of a cranberry extract enriched in total phenols and free, essentially free, or substantially free of sugars, organic acids, sulfur and other contaminants and 90-99% of a Nupro®t prophylaxis paste available from Dentsply International of York, Pa.

Preferred treatment approaches include:

Step 1—dentist chair side gel or swish and swallow;

Step 2—take home, high strength rinse, gum, lozenge and/or toothpaste

Step 3—longer term care at home at lower strength.

Preferably, the products and treatments described above result in from 0.05 to 5 grams of the total phenols are ingested per day.

A preferred method of manufacturing preferred formulations of extracts of cranberries and other fruits and berries containing phenols to produce extracts enriched in total phenols and free, essentially free or substantially free of sugars, acids, sulfur and other contaminants, a crude extract is purified using a brominated polystyrene resin rather than on a conventional polystyrene resin or other resins used in the art provides total phenol-enriched compositions having higher purities.

In one embodiment of the preferred process, an extraction step is accomplished by placing fresh or dried plant material in an appropriate amount of extraction solvent. In one embodiment, the extraction solvent comprises an acidified alcohol solution having about 0-95% ethanol in water and a suitable acid in an amount of about 0-3%, more preferably about 0.006-0.012% by weight. In another embodiment, the extraction solvent comprises an acidified alcohol solution having between about 0-100% methanol in water and between about 0-3% by weight of a suitable acid. Suitable acids that may be used in the extraction step include, but are not limited to, acetic acid (HOAc) or hydrochloric acid (HCl). The addition of an acid to the extraction solvent prevents degradation of the proanthocyanidins and anthocyanins. Thus, in one embodiment the acidic conditions are maintained throughout most of the steps of the process. The plant material is contacted with the extraction solution for an appropriate amount of time at a temperature between about room temperature and 75° C., preferably at 40° C., to form the crude extract. The amount of plant material to extraction solvent used in the extraction process varies between about 2:1 to about 1:20 on a gram to milliliter basis. In one embodiment, the ratio of plant material to extraction solvent is between about 1:4 and 1:8.

The crude extract contains phenolic compounds such as proanthocyanidins, anthocyanins and other phenolics, as well as undesired non-phenolic materials such as sugars, pectin, plant sterols, fatty acids, triglycerides, and other compounds. Solid residue contained in the crude extract is separated from the liquid portion, and the solids are either re-extracted as described above or discarded.

In one embodiment, pectinase is added either to the plant material or to the extraction solvent before or during the extraction process. Alternatively, the pectinase can be added to the crude extract after the extraction process is complete. The pectinase serves to prevent the extract from gelling at any point during or after the extraction process so that it will remain flowable during the column purification. The amount of pectinase added will depend, of course, on the amount of plant material used to prepare the extract. Typically, the pectinase is added in an amount between about 0 and 0.12% by weight of the plant material.

If either an ethanolic or methanolic extraction solvent was used to prepare the crude extract, the crude extract is concentrated until the crude extract contains less than 6% ethanol or methanol, preferably maintaining a temperature of 40° C. or less during concentration. Water is added to dilute the concentrated crude extract, and the diluted crude extract is either concentrated and diluted again with water, or is carried on directly to the next step without performing a second dilution.

The next step involves filtering the crude extract to remove solids that may have precipitated from the crude extract. Various filtration methods may be employed including adding a measured amount of a filter aid such as diatomaceous earth or cellulose to the crude extract. The mixture of crude extract and filter aid is preferably shaken or stirred until homogeneous and filtered through a bed of filter aid. The bed is washed with an aqueous acidic solution, preferably about 0.006% aqueous sulfuric acid.

Other filtration methods that may be used include filtering the crude extract through a bed of sand or a 30 micron polypropylene filter that is preferably covered with glass wool. Yet another filtration method comprises using a bag filter (a bag-shaped cloth filter composed of polyethyl-ene or polypropylene), which may advantageously be placed in-line with the purification column described below. The filters described above are used to remove precipitated solids and are not size exclusion filters.

To isolate the phenolic compounds according to the preferred method, the filtered extract isolated as described above is contacted with a brominated polystyrene adsorbent material capable of releasably adsorbing the phenolic compounds such as proanthocyanidins and anthocyanins, but which retains less of the undesired non-phenolic materials that were present in the filtered extract. A high purity composition enriched in total phenols could be obtained by purifying the filtered extract isolated as above on a brominated polystyrene resin, such as SP-207 (Supelco; Bellafonte, PA), manufactured by Mitsubishi Chemical America. SP-207 resin is a macroporous, brominated styrenic polymeric bead type resin designed for reversed-phase chromatographic applications, and has a particle size distribution between about 250-600 microns and a pore size range between about 100-300 Angstroms. The bromination of the aromatic rings provides increased hydrophobicity to the polystyrene resin, and is designed to provide a resin having increased selectivity for hydrophobic molecules relative to conventional styrene-divinylbenzene polymeric reversed-phase supports. Because of its tight binding properties, brominated polystyrene resin is not typically used in the purification of natural products.

Preferably, the filtered extract isolated as above is loaded onto a column packed with brominated polystyrene resin having a particle size distribution between about 250-600 microns and a pore size range between about 100-300 Angstroms. Note that while use of a with a resin packed into a column is preferred, the resin need not be packed into a column in order to perform the method of this invention. The amount of filtered extract that is loaded onto the column depends on the plant material used to prepare the crude extract. For example, when the crude extract is prepared from bilberries, about 16-30 grams of total phenols may be loaded per liter of resin. As another example, when the crude extract is prepared from blueberries, about 15-45 grams of total phenols may be loaded per liter of resin. When the crude extract is prepared from elderberries, about 15-40 grams of total phenols may be loaded per liter of resin. The filtered extract may be diluted with water prior to loading if the solids concentration in the concentrated crude extract exceeds 200 grams per liter. The fractions eluting during column loading are collected as “fraction 1.”

Subsequent to loading the filtered crude extract onto the resin, undesired non-phenolic materials (e.g., sugars, salts, organic acids, etc.) which have little or no affinity for the adsorbent are eluted from the resin with an aqueous wash solvent comprising at least 0.003% acid such as aqueous sulfuric acid, aqueous acetic acid or aqueous hydrochloric acid. For example, about three column volumes of 0.006% aqueous sulfuric acid or 0.1% aqueous acetic acid can be used to elute the extraneous materials. The eluent is collected as “fraction 2.”

The column is next eluted with a first eluent comprising a polar organic solvent such as about 50 to 70% ethanol/water or about 50 to 90% methanol/water. Typically about 2 to 12 column volumes of eluting solvent are used. In one embodiment, the first eluent contains about 0.003% of an acid such as sulfuric acid, hydrochloric acid or acetic acid. The fraction(s) collected during this elution step are collected as “fraction 3.” “Fraction 3” contains a portion of the phenolic compounds contained in the crude extract and is particularly enriched in anthocyanins and contains proanthocyanidins.

After the majority of the anthocyanins have been eluted from the column, as determined by UV-VIS spectroscopy, the column is eluted with a second eluent comprising a polar organic solvent comprising a greater percentage of ethanol or methanol than the solvent used to elute the anthocyanins. For example, the second eluent may comprise about 50 to 90% ethanol/water or about 75 to 100% methanol/water. The fraction(s) collected during this elution step are collected as “fraction 4.” “Fraction 4” contains an additional portion of the phenolic compounds originally contained in the crude extract and is typically enriched in proanthocyanidins. “Fraction 4” may also contain anthocyanins not isolated during the previous elution step.

Recovery of the phenolic compounds in “fraction 3” and “fraction 4” can be accomplished in any convenient manner such as by evaporation, distillation, freeze-drying, and the like, to provide a total phenol-enriched composition of this invention.

The above-described process is suitable for preparing compositions sufficiently enriched in total phenols for use in the periodontal treatment techniques of the present invention from not only cranberries, but also from a variety of plant materials that contain phenolic compounds including, but not limited to, elderberries, plums, blueberries, bilberries, blackberries, strawberries, red currants, black currants, cherries, raspberries, grapes, hibiscus flowers, bell peppers, beans, peas, red cabbage, purple corn, and violet sweet potatoes. In one embodiment, the enriched compositions of this invention contain at least 10-80% total phenols. In another embodiment, the compositions contain at least 12% total phenols. In yet another embodiment, the compositions contain at least 25% total phenols.

The total phenol-enriched compositions, and in particular the compositions isolated from a combination of “fraction 3,” and “fraction 4,” prepared from fruits and berries in particular produce similar HPLC chromatograms having the characteristic peaks that are not contained in HPLC chromatograms of compositions prepared from plant material other than fruits and berries. When the total phenol-enriched compositions of this invention are analyzed by IR spectrometry, characteristic peaks from the phenolic compounds are also observed.

1. A periodontal treatment, comprising:
contacting the periodontal surfaces with a cranberry extract formulation enriched in total phenols and substantially free of sugars, acids, sulfur and other contaminants; and
maintaining the formulation in contact with the periodontal surfaces.

2. The periodontal treatment of claim 1, wherein the cranberry extract formulation is selected from the group consisting of an aqueous solution, a slurry, a gel, a gum, a lozenge, a toothpaste and a powder.

3. The periodontal treatment of claim 1, wherein the contacting step involves swishing, gel application, gum chewing, and brushing with a toothbrush.

4. The periodontal treatment of claim 1, wherein the cranberry extract formulation enriched in total phenols is essentially free of sugars, acids, sulfur and other contaminants.

5. The periodontal treatment of claim 4, wherein the cranberry extract formulation is selected from the group consisting of an aqueous solution, a slurry, a gel, a gum, a lozenge, a toothpaste and a powder.

6. The periodontal treatment of claim 4, wherein the contacting step involves swishing, gel application, gum chewing, and brushing with a toothbrush.

7. The periodontal treatment of claim 1, wherein the cranberry extract formulation enriched in total phenols is free of sugars, acids, sulfur and other contaminants.

8. The periodontal treatment of claim 7, wherein the cranberry extract formulation is selected from the group consisting of an aqueous solution, a slurry, a gel, a gum, a lozenge, a toothpaste and a powder.

9. The periodontal treatment of claim 7, wherein the contacting step involves swishing, gel application, gum chewing, and brushing with a toothbrush.

10. A periodontal treatment, comprising:
contacting the periodontal surfaces with a fruit or vegetable extract formulation enriched in total phenols and substantially free of sugars, acids, sulfur and other contaminants, wherein the fruit or vegetable is selected from the group consisting of blueberries, bilberries, elderberries, plums, blackberries, strawberries, red currants, black currants, cranberries, cherries, chokecherries, mangosteen, raspberries, grapes, currants, hibiscus flowers, bell peppers, beans, peas, red cabbage, purple corn, and violet sweet potatoes; and
maintaining the formulation in contact with the periodontal surfaces.

11. The periodontal treatment of claim 10, wherein the extract formulation is selected from the group consisting of an aqueous solution, a slurry, a gel, a gum, a lozenge, a toothpaste and a powder.

12. The periodontal treatment of claim 10, wherein the contacting step involves swishing, gel application, gum chewing, and brushing with a toothbrush.

13. The periodontal treatment of claim 10, wherein the extract formulation enriched in total phenols is essentially free of sugars, acids, sulfur and other contaminants.

14. The periodontal treatment of claim 13, wherein the extract formulation is selected from the group consisting of an aqueous solution, a slurry, a gel, a gum, a lozenge, a toothpaste and a powder.

15. The periodontal treatment of claim 13, wherein the contacting step involves swishing, gel application, gum chewing, and brushing with a toothbrush.

16. The periodontal treatment of claim 10, wherein the extract formulation enriched in total phenols is free of sugars, acids, sulfur and other contaminants.

17. The periodontal treatment of claim 16, wherein the extract formulation is selected from the group consisting of an aqueous solution, a slurry, a gel, a gum, a lozenge, a toothpaste and a powder.

18. The periodontal treatment of claim 16, wherein the contacting step involves swishing, gel application, gum chewing, and brushing with a toothbrush.
